(19) United States
(12) Patent Application Publication
(10) Pub. No.: US 2019/0135927 A1
(43) Pub. Date: May 9, 2019

(54) MIGRAINE, HEADACHE, CHRONIC PAIN TREATMENT, AND PROPHYLAXIS OPTIONS

(71) Applicant: BHL PATENT HOLDINGS, LLC, TOWANDA, PA (US)

(72) Inventor: BRUCE H. LEVIN, PHILADELPHIA, PA (US)

(21) Appl. No.: 16/145,978

(22) Filed: Sep. 28, 2018

Related U.S. Application Data
(60) Provisional application No. 62/565,499, filed on Sep. 29, 2017.

(51) Int. Cl. C07K 16/28; A61P 21/00; A61P 25/02; A61P 25/06; A61P 29/00

(52) U.S. Cl. CPC C07K 16/2869; A61P 21/00; C07K 2317/76; A61P 25/06; A61P 29/00; A61P 25/02

(57) ABSTRACT

Disclosed is method comprising administering anti-CGRP medications or monoclonal antibodies to CGRP and/or its receptors with neuroaugmentation to decrease pain associated with headache or chronic pain, for example migraine or cluster headache, or facial pain such as peripheral neuropathy.

### CROSS REFERENCE TO RELATED APPLICATION

This application claims priority from U.S. Provisional Application Ser. No. 62/565,499, filed Sep. 29, 2017, entitled “Improved Migraine, Headache, Chronic Pain Treatment, And Prophylaxis Options”, which is hereby incorporated by reference.

### BACKGROUND OF THE INVENTION

This invention relates to the field of treatment of headache, facial pain and pain syndromes.

There is a great need for improved migraine treatment and prophylaxis options.

In the 2015 Global Burden of Disease Study, migraine was the seventh leading cause of disability globally and the leading neurological cause of disability with direct costs of almost $9 billion and approximately equal secondary costs. Impact on patients' quality of life is severe.

Current popular treatment modalities yield suboptimal results and significant side effects.

### SUMMARY OF THE INVENTION

In one aspect the invention comprises a method comprising administering anti-CGRP medications or monoclonal antibodies to CGRP and/or its receptors with neuroaugmentation to decrease pain associated with headache or chronic pain. Examples of headache include migraine and cluster headache. Examples of chronic pain include facial pain or peripheral neuropathy.

In another aspect, the neuroaugmentation is of the SPG and/or other dorsonasal nerve structures, trigeminal nerve, occipital nerve, facial nerve, Supratrochlear, Supraorbital, infra orbital, infratrochlear, Auriculotemporal or other peripheral nerve or branch of cranial nerve or nerve of the head or face.

In some embodiments wherein the decreased pain relates to episode severity, frequency, duration, dose related or continued use side effects, required doseages, optimizing dosing schedule, cost, expanding patient population qualifications and other issues.

Another aspect of the invention comprises administering anti-CGRP medications or monoclonal antibodies to CGRP and/or its receptors with neuroaugmentation to decrease one or more of either of any neurologic symptoms, morbidity, cumulative changes and or mortality from neurologic pathologies selected from the group consisting of stroke, TIA, CVA, hemmorhage, dementia, tinnitus, vascular ischemia, small vessel ischemia, lacunae state, seizures, and degenerative changes of the brain.

### DETAILED DESCRIPTION

CGRP is a 37 aminoacid vasoactive peptide, it's infusion into migraineurs triggers migraine symptoms identical or very similar to usual migraine episodes. See Durham PL. Calcitonin Gene-Related Peptide (CGRP) and Migraine. Headache. 2006; 46 (Suppl 1):S3-S8.

CGRP transcription is increased with neurogenic inflammation and similar pathologies, and with presence of TNF. Further, alpha TNF alpha1 is also elevated in migraines and increases CGRP promotion. Hence, these increase pro inflammatory mediators.

The duration of increased CGRP synthesis and release correlates with migraine duration.

CGRP is released from cultured Trigeminal nerve cells.

The cell bodies of the Trigeminal ganglia are the main source of CGRP in the Trigeminovascular system.

Migraine medications Triptans decrease both transcription and release of CGRP, and decrease promotor activity to 25-50% of control.

Serum CGRP levels are elevated during migraine attacks and decrease with decreased Sx with Triptan administration.

Trigeminal nerve sensory fiber activation induce neurogenic edema which further increases vasoactive peptide release including CGRP as well TNF alpha, Nerve Growth Factor (which activate Mitogen activated protein kinase MAPKs) etc.

KCl induced Trigeminal neural depolarization, inflammatory cocktail and Capscaicin all increase CGRP release.

Sumatriptan inhibits CGRP release in KCL activated, but not in nonactivated, Trigeminal cultures, but requires higher levels than seen clinically. There are several putative reasons for the latter.

Other 5HT1 agents inhibit release from inflammatory cocktail.

Triptans inhibit NGF and MAPK induced CGRP promotor activity by a cAMP independent increase in Intracellular Ca+2.

Botox A also inhibits CGRP release in activated but not normal Trigeminal cells

There is a possibility of increased intracellular Ca+2 levels accounting for decreased CGRP release via increased activity of okadaic acid sensitive phosphatase.

In series and parallel brainstem and higher level spinal cord centers are directly and cross activated increasing snowballing of the cascade.

These pathologies result in a period of relative hyperexcitabilty where the headache and related symptoms increase and repropropagation of the headache can occur following treatment.

Because the period of hyperexcitabilty often outlives the period of effective treatment, rebound is common.

In a prior patent application I showed that the duration of activity of Lidocaine was too short to break many migraine cycles, resulting in suboptimal treatment results or rebound.

That patent held that utilizing a long acting local anesthetic agent to block 1 or more dorsonasal nerve structures would result in an interruption of the pathological neuronal pathways for a time period which exceeded the period of neuronal hyperactivity in key pathways. Thus, using longer acting local anesthetics can more effectively treat migraine episodes and without recurrence.

Further, with ongoing treatment, in several patients there was decrease in one or more of episode frequency, duration, distribution, intensity, and or degree of associated co-symptomatolgies.

Initially 6 CGRP antagonists were identified and some trials were quite successful in treating migraines, but two of these agents were dropped because of associated hepatic toxicity (i.e., Telcagepant which decreased migraine days compared with placebo at one month with LFTs up to 3×nl) and 1 was dropped because of formulation issues. Ubrogepant has consideration for acute treatment and Abrogepant for prophylaxis.

Monoclonal Ab to CGRP including LY2951742, ALD-403, and LBR-101/TEV-48125 or to CGRP receptor, AMG334, have shown a good safety profile and efficacy, noteably in high frequency episode and chronic migraine, but are not effective for all migraineurs and must be administered IV or SubQ but in monthly or quarterly intervals to improve compliance. (Intern Emerg Med. 2016 December; 11(8): 1045-1057. Epub 2016 Jun. 23. Anti-CGRP monoclonal antibodies in migraine: current perspectives).

In Curr Treat Options Neurol. 2017; 19(8): 27, the following is reported regarding Galcanezumab (LY2951742): The first phase II clinical trial results to be published were for Eli-Lilly's galcanezumab [38]. This study randomized patients with episodic migraine (4 to 14 headache days in 4-week baseline period) to galcanezumab 150 mg subcutaneously versus placebo every 2 weeks for 12 weeks. Primary efficacy endpoint was the change in number of migraine days during the third 4-week treatment period (weeks 9-12) compared to the baseline period. The mean change in migraine headache days was significantly different in the galcanezumab group compared to the placebo group (−4.2 versus −3.0 days, respectively; least squares mean difference 1.2, p=0.0030).

Eptinezumab (ALD-403): Alder Biopharmaceuticals took a slightly different approach with eptinezumab, reasoning that intravenous administration would result in rapidly efficacious dosing with immediate physiological effect. Patients with episodic migraine (5 to 14 headache days in 4-week baseline period) were randomized to either a single dose of monthly intravenous eptinezumab 1000 mg or placebo. The primary efficacy endpoint was the change in number of migraine days during weeks 5-8 compared to the baseline period. With a one-sided p value (pre-specified), eptinezumab resulted in significantly fewer migraine days compared to placebo (−5.6 versus −4.6 days, respectively; difference 1.0, p=0.0306) [39].

Fremanezumab (TEV-48215 or LBR-101): Teva Pharmaceuticals investigated fremanezumab in two separate trials for patients with either high-frequency episodic migraine or chronic migraine [40, 41]. Patients with 8 to 14 headache days in 4-week baseline period were randomized to subcutaneous injections of either fremanezumab 225 or 675 mg or placebo every 4 weeks for 12 weeks. Primary efficacy endpoint was the change in number of migraine days during the third 4-week treatment period (weeks 9-12) compared to the baseline period. The least square mean reduction in migraine days was significantly greater compared to placebo for both the fremanezumab 225 mg (−6.27 versus −3.46 days; difference 2.81 days, p<0.0001) and 675 mg doses (−6.09 versus −3.46 days; difference 2.64 days, p<0.0001). Of note, in contrast to the previous two studies, patients were not excluded for use of a migraine preventive; use of one preventive was allowed provided the dose had been stable for 2 months prior to screening.

Erenumab (AMG-334): Lastly, Amgen has developed a monoclonal antibody against the CGRP receptor, erenumab, in contrast to the other three antibodies that are targeted at the CGRP molecule itself. Patients with episodic migraine (4 to 14 headache days in 4-week baseline period) were randomized to either placebo or one of three doses of erenumab (7, 14, or 70 mg) subcutaneously every 4 weeks for 12 weeks. Primary efficacy endpoint was the change in number of migraine days during the third 4-week treatment period (weeks 9-12) compared to the baseline period. The least square mean change in migraine headache days was significantly different from the placebo group only for the highest dose, erenumab 70 mg (−3.4 versus −2.3 days; difference 1.1 days, p=0.021) [42].

For the chronic migraine trial, patients were randomized to either placebo or one of two fremanezumab doses given every 4 weeks for 12 weeks: 675 mg loading dose followed by two 225 mg doses or three doses of 900 mg. Primary efficacy endpoint was change in hours of headache of any severity during the third 4-week treatment period (weeks 9-12) compared to the baseline period. The least square mean reduction in headache hours of any severity was significantly greater compared to placebo for both the fremanezumab 675 mg/225 mg/225 mg (−59.8 versus −37.1 h; difference 22.7 h, p=0.0386) and 900 mg doses (−67.5 versus −37.1 h; difference 30.4 h, p=0.0057). Use of up to two preventives was permitted provided the doses were stable for at least 3 months. Of note, although chronic migraine is defined as 15 or more days of headache per month, the mean number of headache days per month in the study population was approximately 16. Thus, the results may not be generalizable to patients with daily or near daily headache.

In those patients who do not respond completely to SPG block, Triptan or other therapy is often more effective. In this patient population, CGRP therapy would be enhanced by stimulation of sensory branches of the Supratrochlear, Supraorbital, Auriculotemporal nerves, or by stimulation of occipital nerves or cervical cord level, or by Trigeminal stimulation. It is likely that this stim application would function through the sensory limb primarily decreasing CGRP release more that transcription. However, even here multiple pathways are involved, particularly with Trigeminal stimulation.

CGRP receptors are found outside of the nervous and vascular systems, including in the adrenal glands, kidneys, pancreas, and bone. The effect of chronic CGRP antagonism on other organs is unknown.

Monoclonal antibodies are large molecules that cross the blood-brain barrier in a small ratio of 1:1000 [46], although in individual patients, the ratio may favor penetration more [47]. Thus, their site of action in migraine prevention is unclear.

Patients who failed 2 types of prophylactic categories were excluded, hence it is unknown what benefit CGRP monoclonal Ab administration would yield for this population.

Hence, there is an unmet need to optimize results obtained with anti-CGRP and or receptor monoclonal Ab or other antagonist therapies in terms of results or endpoints including but not limited to episode severity, frequency, duration, dose related or continued use side effects, required doseages, optimizing dosing schedule, cost, expanding patient population qualifications and other issues.

Combination therapy utilizing anti-CGRP agents or monoclonal or other antibodies to CGRP and/or one or more of its receptor(s) with stimulation therapies targeting a branch or branches of or more of the SPG, and or other Dorsonasal neural structures, Trigeminal nerve, Peripheral Branches of the Trigeminal nerve, Occipital or higher cervical spinal cord levels will produce additive or synergistic effects against one or more of a given patient's migraine headache frequency, duration, distribution, intensity, quality, or presence of associated significant symptoms.

Triptans (and other trialed current headache remedies) which had not been helpful, became helpful in terms of decreasing headache severity and frequency following partially successful SPG blocks.

SPG block decreases neurogenic edema, and decreases facial edema, facial allodynia and hyperalgesia, associated symptoms such as nausea/vomiting photo and phonophobia, and decreases episode frequency, and duration and severity. Subsequent treatment with Triptans has greater efficacy in between SPG blocks. SPG stimulation displays similar benefits. (See before and after photos or videos of blocked or neuroaugmented patients.)

SPG blocks also decrease neurogenic edema, as do sympathetic and other blocks used to decrease symptoms of peripheral neuropathies and sympathetic mediated disorders which share similar pathophysiologic pathways, and which clearly decrease visible neurogenic edema, discoloration, allodynia and pain of affected limbs.

SPG block mediated changes of Blood Brain Barrier permeability from SPG stim will allow an increased passage of the large monoclonal antibody through the BBB increasing efficacy.

Further, intranasal and preferably dorsonasal administration targeting an area of or near a dorsonasal or superonasal neuronal or vascular structures or tissues, of anti-CGRP medications or monoclonal antibodies to CGRP and/or its receptors will decrease onset time and allow increased efficacy for acute or subacute migraine, other headache, or facial pain episodes, as well as increasing efficacy of prophylactic use of these agents.

Adding a composition, pharmaceutical, or biologic or other, to increase tissue, mucosal, vascular, or neuronal, CNS, or CSF uptake with improved efficacy. This can decrease the burden of IV or IM or Subcutaneous administration.

This is true of all antibody or larger protein or aminoacid or carbohydrate agents or compositions.

Treatment of migraine, cluster or other headache with either or both of Neurostimulation of the SPG and/or other dorsonasal nerve structures trigeminal nerve, occipital nerve, facial nerve, Supratrochlear, Supraorbital, infra orbital, infratrochlear, Auriculotemporal or other peripheral nerve or branch of cranial nerve or nerve of the head or face, or CGRP or receptor antibody or anti-CGRP agent will lower one or more of either of any neurologic symptoms, morbidity, cumulative changes and or mortality from neurologic pathologies including but not limited to stroke, TIA, CVA, hemmorhage, dementia, tinnitus, microvascular or other small vessel ischemia, lacunae state, seizures or degenerative changes.

While this invention has been disclosed with reference to specific embodiments, it is apparent that other embodiments and variations of this invention may be devised by others skilled in the art without departing from the true spirit and scope of the invention.

The appended claims are intended to be construed to include all such embodiments and equivalent variations.

What is claimed is:

1. A method comprising administering anti-CGRP medications or monoclonal antibodies to CGRP and/or its receptors with neuroaugmentation to decrease pain associated with headache or chronic pain.

2. The method of claim 1 where in the headache is migraine or cluster headache.

3. The method of claim 1 wherein headache or chronic pain is facial pain.

4. The method of claim 1 wherein the chronic pain involves peripheral neuropathy, neuropathic pain, or sympathetic mediated pain syndromes.

5. The method of claim 1 wherein the neuroaugmentation is of the SPG and/or other dorsonasal nerve structures, trigeminal nerve, occipital nerve, facial nerve, Supratrochlear, Supraorbital, Infraorbital, Infratrochlear, Auriculotemporal or other peripheral nerve, or one or more branches of cranial nerve or nerve of the head or face, or Occipital or cervical spinal cord levels.

6. The method of claim 1 wherein the decreased pain relates to episode severity, frequency, duration of symptoms.

7. The method of claim 1 wherein benefits included minimizing dose related or continued use side effects, decreasing frequency of required medication doseages, optimizing dosing schedule, decreasing medication cost, expanding patient population qualifications and other issues relating to route and method of delivery of CGRP medications or monoclonal antibodies to CGRP and/or its receptors.

8. A method comprising administering anti-CGRP medications or monoclonal antibodies to CGRP and/or its receptors with neuroaugmentation to decrease one or more of either of any neurologic symptoms, morbidity, cumulative changes and or mortality from neurologic pathologies selected from the group consisting of stroke, TIA, CVA, hemmorhage, dementia, tinnitus, vascular ischemia, small vessel ischemia, lacunae state, seizures, and degenerative changes of the brain.
